(19) United States
(12) Patent Application Publication
(10) Pub. No.: US 2008/0027499 A1
(43) Pub. Date: Jan. 31, 2008

(54) INTEGRATED HEALTH CARE HOME COMMUNICATION AND MONITORING SYSTEM

(76) Inventors: Muralidharan Srivathsa, Pittsburgh, PA (US); Alan H. Smythe, White Bear Lake, MN (US); Kenneth P. Hoyme, Plymouth, MN (US); Gaurav Rana, Shoreview, MN (US)

Correspondence Address: SCHWEGMAN, LUNDBERG & WOESSNER, P.A., P.O. BOX 2938, MINNEAPOLIS, MN, 55402, US

(21) Appl. No.: 11/460,786

(22) Filed: Jul. 28, 2006

(51) Int. Cl. A61N 1/00

(52) U.S. Cl. 607/32

(57) ABSTRACT

This document discusses, among other things, systems and method for integrated health care home communication and monitoring. A method comprising: communicating, from a remote server to an external interface device that communicates with a medical device in use by a patient, a patient communication to be received by the patient; and receiving, from the external interface device, at the remote server, a patient acknowledgement to the patient communication, the patient acknowledgement indicative of the patient communication having been delivered by the external interface device to the patient and acknowledging that the patient has received the patient communication.

### TECHNICAL FIELD

This patent document pertains generally to implantable medical devices, and more particularly, but not by way of limitation, to integrated health care home communication and monitoring.

### BACKGROUND

Implantable medical devices (IMDs), including cardiac rhythm management devices such as pacemakers and implantable cardioverter/defibrillators, typically have the capability to communicate data with an external device, such as an external programmer, via wireless telemetry, such as a radio-frequency (RF) telemetry link. While an external programmer is typically provided to program and modify the operating parameters of an IMD, modern IMDs also include the capability for bidirectional communication so that information, such as physiological data, can be transmitted to the programmer. Home health care monitoring systems can also communicate with the IMD and collect the patient data. In addition, some monitoring systems can also collect other objective or subjective data using additional external sensors, such as a blood pressure cuff, a weight scale, or a specialized device that prompts the patient with questions regarding their health state. Home health care monitoring systems can communicate with a centralized system, either directly or through a networked system. Centralized systems provide an efficient mode for physicians and other medical practitioners to view patient data and communicate with their patients and with the medical community at large.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic view illustrating portions of a system that enables physician-patient communication.

FIG. 2 is a flowchart illustrating generally a method for communicating with a patient's external interface device.

FIG. 3 is a flowchart illustrating generally a method for receiving and reporting information received from a remote server system at a health monitor.

FIG. 4 is an illustration of an example of a graphical display on a patient's health monitor.

FIG. 5 is a diagram illustrating communication between a health monitor and a remote server system.

FIG. 6 is an illustration of a report showing several message transactions.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

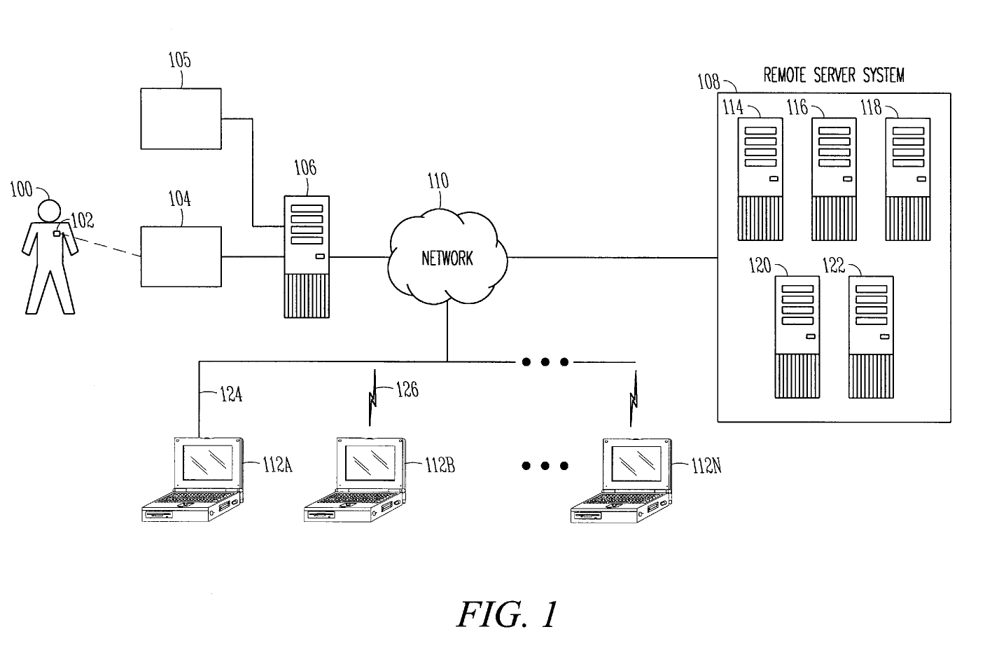

FIG. 1 is a schematic view illustrating portions of a system that enables physician-patient communication. In the example of FIG. 1, a patient 100 is provided with an IMD 102, such as a cardiac rhythm management or other device. In some examples, the IMD 102 is capable of sensing physiological data and storing such data for later communication. The IMD 102 communicates with an external transceiver 104. Typically, the IMD 102 receives commands from the transceiver 104. In some examples, the IMD 102 can transfer one or more patient indications to the transceiver 104. In other examples, the IMD 102 communicates signal data to the transceiver 104, which may then communicate the signal data to another computer for processing. In a further example, the IMD 102 communicates status data to the transceiver 104 for storage or to be communicated to another computer for processing or storage. Typically, the transceiver 104 is located in close proximity to the patient 100. The transceiver 104 may be included within a personal computer or a specialized device, such as a programmer or a home monitoring device. In one example, the transceiver 104 is a hand-held device that is capable of connecting to a health monitor 106. Typically, the connection can be made using a hard-wired connection (e.g., serial, USB, Firewire) or a wireless connection (e.g., RF, IR). In some examples, the health monitor 106 is a specialized device or a personal computer. In an example, the transceiver 104 and health monitor 106 are integrated into a single device.

One or more external devices 105 can be used to measure physiological data. Such devices 105 may include a multitude of devices to measure data relating to the human body, including temperature (e.g., a thermometer), blood pressure (e.g., a sphygmomanometer), blood characteristics (e.g., glucose level), body weight, physical strength, mental acuity, diet, heart characteristics, and relative geographic position (e.g., a Global Positioning System (“GPS”)). An external device 105 can also include one or more environmental sensors. The external devices 105 can be placed in a variety of geographic locations (in close proximity to patient or distributed throughout a population) and can record non-patient specific characteristics such as, for example, temperature, air quality, humidity, carbon monoxide level, oxygen level, barometric pressure, light intensity, and sound.

In certain examples, the health monitor 106 is adapted to communicate with a remote server system 108. Data, such as signal data or status data, which was collected from the IMD 102 or external device 105, can be communicated to the remote server system 108 for storage or processing. The remote server system 108 in some examples is located and managed by a health care provider (e.g., a clinic or hospital) or health information provider such that patients located at home, at a clinic or hospital, or elsewhere may automatically connect and communicate with the remote server system 108. The communication link between the health monitor 106 and the remote server system 108 may be made through a POTS (plain old telephone system) dialup modem connection to an ISP (internet service provider) or directly to a wide-area telecommunications or computer network 110, such as the Internet. In some examples, computer network 110 includes one or more of a wide-area network (WAN), a local-area network (LAN), or a private network. In some examples, the remote server system 108 comprises one or more computers, such as a database server 114, a network server 116, a file server 118, an application server 120 or a web server 122. In certain examples, one or more terminals 112A, 112B, . . . , 112N are connected to the remote server system 108 via the telecommunications or computer network 110. The terminals 112 are communicatively coupled to the network 110 using a wired 124 and/or a wireless connection 126. Typically, a user may connect to the remote server system 108 using a terminal 112, such as to query a patient's personal data or medical history, to initiate commands that administer therapy or program the transceiver 104, or to provide notes or comments that are recorded in the remote server system 108 and optionally communicated to the patient 100.

Other patient monitor configurations are described in commonly assigned U.S. Pat. No. 6,978,182, entitled “ADVANCED PATIENT MANAGEMENT SYSTEM INCLUDING INTERROGATOR/TRANSCEIVER UNIT,” filed on Dec. 27, 2002, which is hereby incorporated by reference in its entirety.

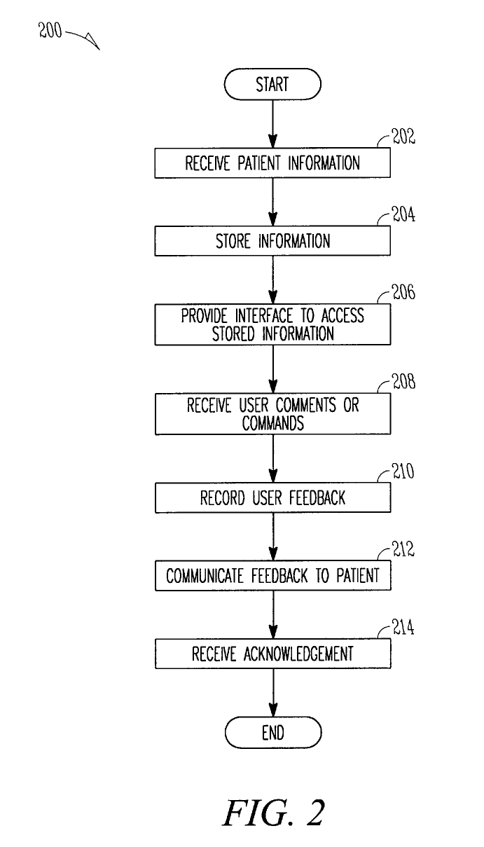

FIG. 2 is a flowchart illustrating generally a method 200 for communicating with a patient's external interface device. In some examples, the patient's external interface device is a health monitor 106, as described in FIG. 1. At 202, a computer at the remote server system 108 receives patient information. In some examples, the information is received from a health monitor 106. In some examples, the information is received directly from an IMD 102 or a transceiver 104. In some examples, the patient information is physiological data obtained from one or more internal (e.g., 102) or external sensors (e.g., 105). In other examples, the patient information can include information such as alerts, patient queries, or summary information, for example summarizing detailed sensor data.

At 204, the information is stored for later retrieval by one or more servers in the remote server system 108. For example, physiological sensor data can be stored in a patient medical history database in the database server 114.

At 206, an interface is provided to a user of the remote server system 108 (e.g., clinician or physician) such that the user can access information contained within the remote server system 108. In one example, the user-interface is a web-based interface constructed using a combination of the web server 122, the database server 114, and other servers in the remote server system 108.

Using the user-interface, the user can access the remote server system 108 and retrieve the patient information. In response, the user may decide to provide comments or feedback based on the patient information. At 208, the user's feedback is received. In some examples, the feedback may include commands, such as reprogramming instructions for the patient's IMD or other medical devices, to be communicated to the patient's health monitor 106. In various embodiments, user comments may be in text, audio, or other multimedia formats.

At 210, one or more aspects of the user's interaction with the remote server system 108 are recorded. For example, if the user provided comments, then they can be recorded along with pertinent supplemental information, such as a date time stamp, an importance or priority.

At 212, information received from the user is communicated to the patient via, for example, the health monitor 106. In some examples, the transmission is recorded for auditing and future reference.

At 214, an acknowledgement is received and recorded. In various examples, the acknowledgement indicates that the message was delivered to a patient or read by a patient or both. In an example, a patient's action, such as opening a message or positively acknowledging the receipt of the message, generates a return message, such as a patient acknowledgement, which may then be stored by the remote server system 108 and then communicated to a user, such as a clinician. In one example, a device at the patient's end of the communication, such as the health monitor 106, sends a device-level acknowledgement upon receipt of a message from the remote server system 108.

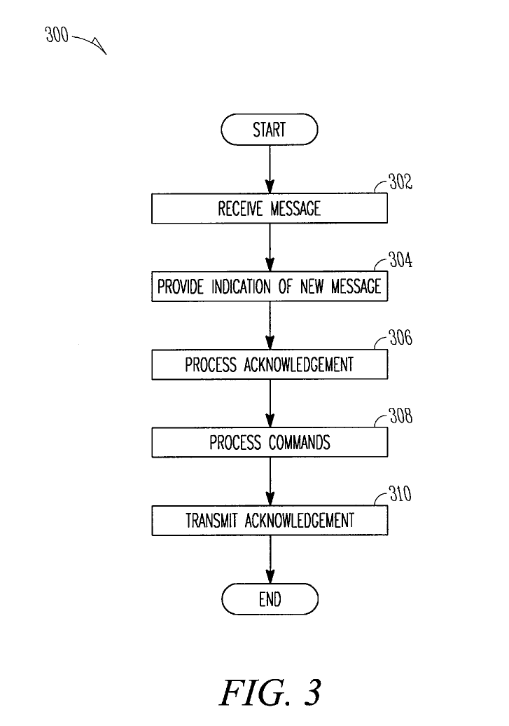

FIG. 3 is a flowchart illustrating generally a method 300 for receiving and reporting information received from a remote server system 108 at a health monitor 106. At 302, the information, in the form of one or more messages, is received by a health monitor 106. In some examples, the health monitor 106 periodically or recurrently connects to the remote server system 108. In other examples, the health monitor 106 is permanently connect to the network 110 and can communicate with the remote server system 108 at any time. In other examples, the patient 100 may initiate the connection and message retrieval. While connected with the remote server system 108, the health monitor 106 can retrieve new messages or communications. In certain examples, such a message may come from the patient's health care professional. In other examples, such a message may come from an IMD manufacturer. In various examples, messages can include different types, such as an informational message, a query message, or a device programming message.

At 304, one or more indications of new messages are provided to a user, such as a patient. In an example, a graphical display is provided to the patient 100 and an icon or other graphical indication is displayed to indicate one or more new messages. In other examples, sounds, lights, vibration, or other techniques are used to alert a patient 100 of new messages. Messages may be presented in a chronological order, grouped or ordered by message type, message format, message origin, message priority, where the ordering or grouping is indicated by descriptive text, text color, text face, or other indicia in various examples. Message types may include device programming messages, informational messages, or query messages. Message formats may include voice or text. Messages may include one or more priority levels. For example, a clinician may want to send an important or high-priority message for the patient to review quickly. In such an example, the high-priority message may be displayed first or before other lower-priority messages.

At 306, one or more patient acknowledgements are processed by the health monitor 106. In an example, when a patient 100 accesses a message, a patient acknowledgement is automatically generated at the health monitor 106 and sent or queued for later sending to the remote server system 108. In an example, the patient 100 is prompted with a dialog box asking for confirmation or permission to send a patient acknowledgement to the remote server system 108. In another example, a voice recording is provided to the patient 100 and the patient acknowledgement is automatically or manually generated after the message has been fully played for the patient 100. In an example, the message to which the patient 100 is responding is a notification of one or more changes to the programming of the patient's IMD. In some examples, a patient acknowledgement serves as a patient consent, such as to perform IMD reprogramming or the like.

At 308, messages containing commands are processed. In some examples, commands are contained in one or more messages received by the health monitor 106. In an example, command messages are processed when the IMD 102 is within communication range of the transceiver 104 and commands are issued via a communication link between transceiver 104 and IMD 102. In an example, the processing includes connecting with the implanted device 102 and modifying one or more parameters or programs to alter the implanted device's operation. In some examples, commands are processed after receiving a patient acknowledgement or patient consent via the health monitor 106. In other examples, commands are processed upon receipt of the container message, such that if a patient is in communication range, programming instructions are provided to the patient's IMD 102 to reprogram or modify the IMD's function.

At 310, patient acknowledgements are transmitted to the remote server system 108, where they are received and recorded. In an example, patient acknowledgements are queued for later sending, such as when the health monitor 106 periodically or regularly connects with the remote server system 108. In an example, when an acknowledgement message is received at the remote server system 108, the physician or other user who sent or is associated with the original message is notified of the acknowledgement.

For example, a physician may send a message to a patient using the remote server system 108. Upon receiving and reading the message, the patient at the health monitor 106 generates an acknowledgement, which is sent from to the remote server system 108 and recorded. The physician may then access the remote server system 108 to verify that the patient received and viewed the message sent. In an alternative example, the remote server system 108 may notify the physician of the acknowledgement using a notification messaging system, such as a pager message, automated cell phone call, text messaging, email, or the like. In some examples, only certain types or classes of messages may cause the remote server system 108 to notify a user (e.g., a clinician) of the acknowledgement message. For example, if a patient has failed to read or acknowledge a physician message for a threshold time, then a notification of the failure condition may be sent to the physician, whereas successful acknowledgements may be recorded for later reference, but would not generate a notification message to the physician.

In another example, the remote server system 108 may automatically generate a reminder message, such as to remind a patient to take their daily medicine, and upon receiving an acknowledgement indicating the receipt of the message by the patient, the patient's physician may be notified that the patient received and acknowledged the daily reminder. If, however, the patient neglected to acknowledge receipt or was unable to acknowledge receipt, the physician may be notified after a threshold time period, such as that the patient may not have taken their daily dosage of medicine. In examples, the notification may be generated at the remote server system 108 after detecting that an acknowledgement has not been received in a threshold time or the notification may be generated at the health monitor 106 and communicated to the physician via the remote server system 108.

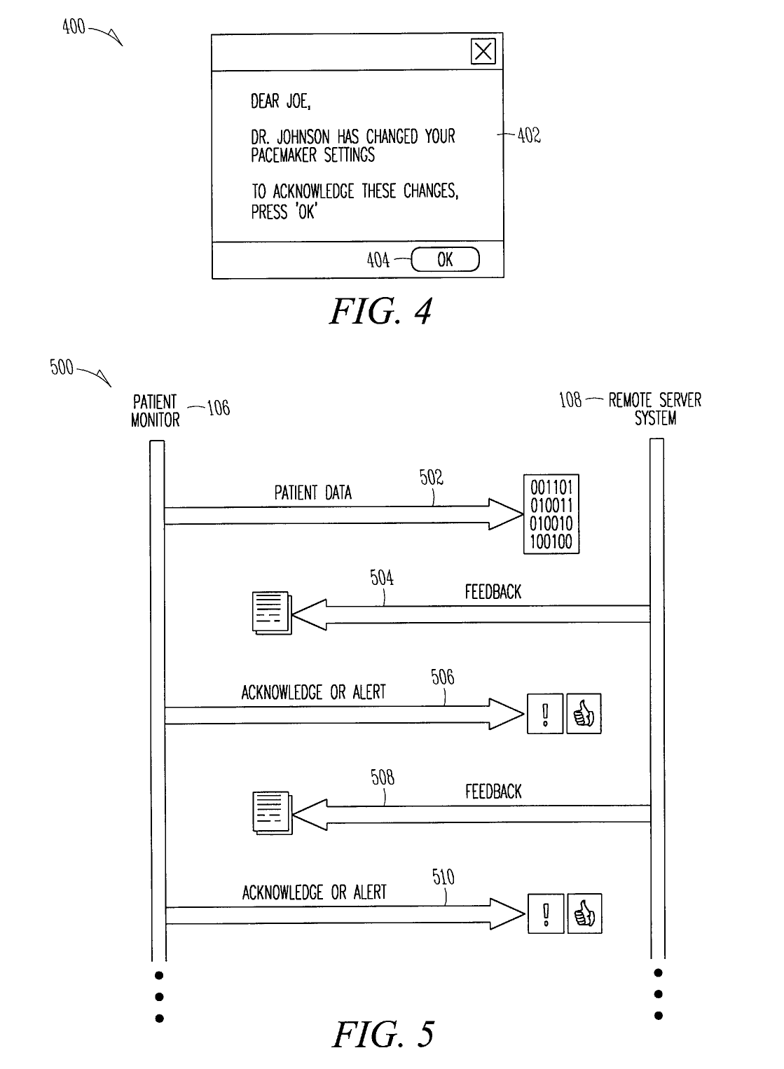

FIG. 4 is an illustration of an example of a graphical display on a patient's health monitor 106. In such a display, the patient 100 can be provided with a dialog box 400 with a message 402 to the patient 100 from a physician or health professional. The dialog box 400 includes an input mechanism 404, such as an “OK” icon, which when activated by the patient 100 will generate an acknowledgement message sent to and logged by a server, such as in the remote server system 108. In some examples, a patient may consent to an action, such as reprogramming the patient's IMD, by activating the “OK” button.

In some examples, the patient must authenticate the acknowledgement before it is sent. For example, the patient can log in to the health monitor 106 using a username and password combination uniquely identifying the patient and that is preferably only known by the patient. In another example, the patient can use an auxiliary device to read biometric information, such as a retinal scanner, fingerprint scanner, voice recognition device, or the like, to authenticate the patient's identity before sending the acknowledgement. An indication of the authentication may be sent along with the acknowledgement message to the remote server system 108 for logging. In another example, the patient can use the auxiliary device to provide an authenticated acknowledgement, which is then communicated to the remote server system 108. In some examples, the remote server system 108 detects and records the authentication as part of the acknowledgement for future reference.

In an example, if the patient decides not to accept the actions of a message or if the patient does not acknowledge a message, then an alert is generated and communicated to the remote server system 108. The alert can be a general alert indicating that the patient has not yet received the message, or may indicate that the patient received the message, but did not acknowledge receipt, or it may indicate that the patient actually declined a therapy modification (e.g., IMD reprogramming). In some examples, an alert is automatically generated after a threshold event, such as a time period or a number of times a patient has viewed a message without indicating acknowledgement. For example, a display on the health monitor 106 may show the patient 100 the subject or message type of each message and allow the patient 100 to select a message to access so the patient 100 can view the contents. If a patient neglects to open a particular message after it is presented several times, an alert can be generated and delivered to the remote server system 108. In such a case, a physician or other person can then follow up with the patient.

FIG. 5 is a diagram illustrating communication 500 between a health monitor 106 and a remote server system 108. Data 502 is sent from the health monitor 106. The data 502 can include raw sensor data from an IMD, data collected from external monitoring devices, patient questions, device data (e.g., status or alert information), or summary data.

After reviewing the data 502, a physician or other health professional may compose a responsive communication. The remote server system 108 communicates one or more responsive messages 504 to the health monitor 106, either in response to the received patient data 502 or otherwise. The responsive messages 504 can be one or more of an information message, a request for more information, a command or instruction for a patient device, or any other type of message that a physician or health professional may communicate with a patient, the patient's IMD or other personal medical device, or the patient's health monitor 106. In addition, the responsive messages 504 may include system-generated responses, such as an informational message informing the patient of an expected response time.

In an example, a return message 506 is communicated from health monitor 106 to the remote server system 108. In some examples, the return message 506 includes an acknowledgement or an alert. For example, if the patient fails to respond (e.g., acknowledge receipt) to the responsive message 504, then an alert may be generated and automatically sent as the return message 506. In some examples, the return message 506 contains patient authentication information, indicating that the patient authorized the message, such as to acknowledge receipt of the message 504.

In response to, or independent of the return message 506, a physician or health professional message 508 is sent to the health monitor 106. The message 508 may or may not be related to the original message 502. The physician-generated message 508 can be of a similar type of message as that communicated in 504.

When the patient reads or acknowledges the message 508, an acknowledgement message 510 is generated and delivered to the remote server system 108. Alternatively, if the patient neglects to read or acknowledge the message 508, then an alert 510 can be generated and communicated to the remote server system 108. As described above, alerts can be generated based on a patient's action or inaction. Other types of alerts can also be automatically delivered, such as when the health monitor 106 is unable to receive a physician message sent by the remote server system 108. This may happen due to a lack of memory at the health monitor 106, a network or other hardware failure, or an incorrect addressing of the message from the remote server system 108. In some examples, the delivery failure is recorded at the remote server system 108 in a similar manner as an acknowledgement.

In some examples, a device-level acknowledgement is communicated from the patient's health monitor 106 to the remote server system 108 after receipt of a physician message. The device-level acknowledgement may be in addition to or in lieu of any user-level (e.g., patient) acknowledgement message. For example, a physician can use an interface at the remote server system 108 to provide a message to a patient. When the physician's message is delivered to the patient's health monitor 106, a device-level acknowledgement is automatically generated by the health monitor 106 and sent to the remote server system 108 in response. The device-level acknowledgement can be recorded by the remote server system 108 in a similar manner as a user-level acknowledgement. The device-level acknowledgement can then serve as proof that the patient's device (e.g., health monitor 106, or IMD 102) successfully received the physician's message from the remote server system 108. One or more thresholds based on the physical receipt of the message by the health monitor 106 can be established such that if a patient fails to open the received message in a timely manner, an alert is generated and communicated to the remote server system 108.

In some examples, one or more reports can be generated, such as by the remote server system 108. In various examples, reports may be related to a single patient or groups of patients, such as patients under a particular physician's care. Reports may include date ranges, time spans, or other temporal periods. Reports may be restricted to one or more message types.

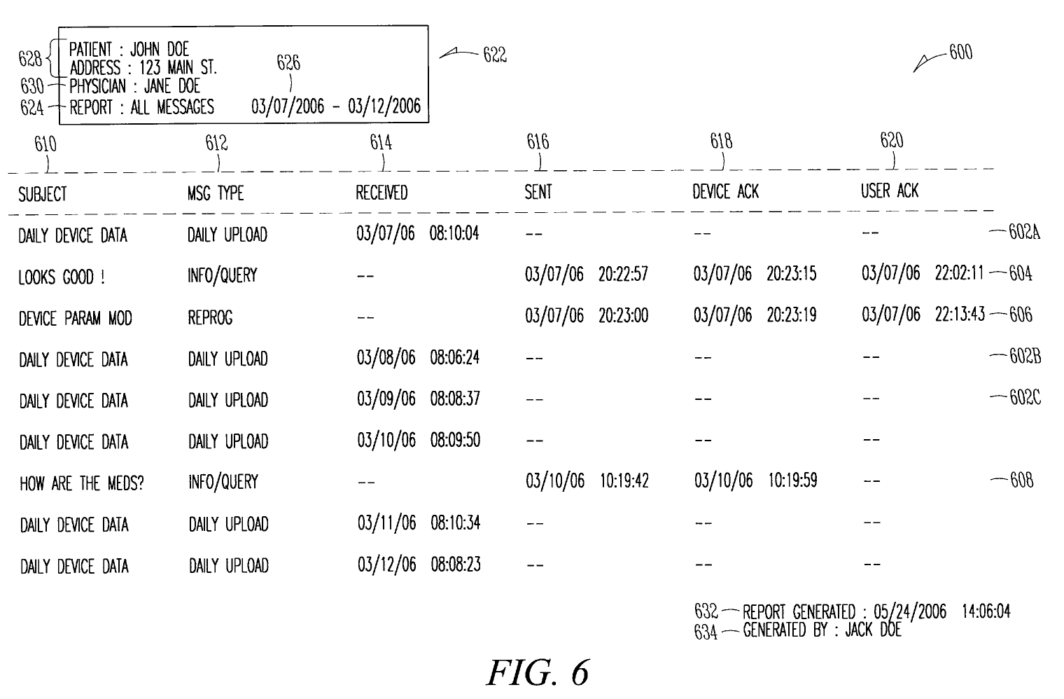

FIG. 6 is an illustration of a report 600 showing several message transactions for a particular patient. In FIG. 6, several messages are illustrated, including a daily upload message 602A, 602B, 602C, an informational message 604, a device programming message 606, and a query message 608. In the report 600, the information is organized into several columns including a subject 610, message type 612, and four timestamp columns, including a received 614, sent 616, device ack (i.e., acknowledge) 618, and user ack 620 timestamps. In some examples, more or fewer columns are included in a report, such as a “from” column, identifying the sender, or a “status” column, indicating a current alert status of the message. The report 600 also includes a header portion 622 that may include such information as a title 624, a date range 626, and other identifying or organizational information, such as a patient's name and contact information 628, the primary care physician 630, the time the report was generated 632, and who generated the report 634.

It is to be understood that the above description is intended to be illustrative and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

For the purposes of this specification, the term “machine-readable medium” or “computer-readable medium” shall be taken to include any medium which is capable of storing or encoding a sequence of instructions for execution by the machine and that cause the machine to perform any one of the methodologies of the inventive subject matter. The terms “machine-readable medium” or “computer-readable medium” shall accordingly be taken to include, but not be limited to, solid-state memories, optical and magnetic disks, and carrier wave signals. Further, it will be appreciated that the software could be distributed across multiple machines or storage media, which may include the machine-readable medium.

Method embodiments described herein may be computer-implemented. Some embodiments may include computer-readable media encoded with a computer program (e.g., software), which includes instructions operable to cause an electronic device to perform methods of various embodiments. A software implementation (or computer-implemented method) may include microcode, assembly language code, or a higher-level language code, which further may include computer readable instructions for performing various methods. The code may form portions of computer program products. Further, the code may be tangibly stored on one or more volatile or nonvolatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The foregoing description of specific embodiments reveals the general nature of the inventive subject matter sufficiently that others can, by applying current knowledge, readily modify and/or adapt it for various applications without departing from the generic concept. Therefore, such adaptations and modifications are within the meaning and range of equivalents of the disclosed embodiments. The phraseology or terminology employed herein is for the purpose of description and not of limitation. Accordingly, the inventive subject matter embraces all such alternatives, modifications, equivalents and variations as fall within the spirit and broad scope of the appended claims.

The Abstract is provided to comply with 37 C.F.R. §1.72(b), which requires that it allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment.

What is claimed is:

1. A machine-assisted method comprising:
communicating, from a remote server to an external interface device that communicates with a medical device in use by a patient, a patient communication to be received by the patient; and
receiving, from the external interface device, at the remote server, a patient acknowledgement to the patient communication, the patient acknowledgement indicative of the patient communication having been delivered by the external interface device to the patient and acknowledging that the patient has received the patient communication.

2. The method of claim 1, comprising recording, by the remote server, documentary evidence of the patient's receipt of the patient communication.

3. The method of claim 1, comprising receiving, at the remote server, physiological information from the external interface device, at least some of the physiological information having been obtained by the external interface device from the medical device, and wherein the patient communication is generated at least in part in response to the physiological information.

4. The method of claim 3, wherein the patient communication is generated automatically at least in part in response to the physiological information.

5. The method of claim 1, comprising receiving, at the remote server, medical device status information from the external interface device, wherein the medical device status information is related to the medical device in use by the patient.

6. The method of claim 1, comprising:
providing to a user of the remote server information from the external interface device received by the remote server; and
receiving from the user information to be included in the patient communication.

7. The method of claim 1, comprising receiving, at the remote server, a device-level acknowledgement, the device-level acknowledgement indicative of the patient communication having been delivered by the remote server to the external interface device.

8. The method of claim 7, comprising recording, by the remote server, information about the device-level acknowledgement.

9. The method of claim 1, comprising notifying a user of the remote server of receipt of the patient acknowledgement.

10. The method of claim 1, comprising generating an alert if the patient acknowledgement is not received within a specified time period.

11. The method of claim 10, comprising communicating the alert to a user of the remote server.

12. The method of claim 10, comprising communicating the alert to the patient.

13. The method of claim 1, wherein the patient communication is an automatically generated response based, at least in part, on the information from the external interface device.

14. The method of claim 1, wherein the response communication includes one or more of: an email, a pager message, a voice message, a text message, or an electronically transmittable message.

15. The method of claim 1, wherein the medical device in use by the patient is an implanted medical device.

16. A machine-assisted method comprising:
receiving, at an external interface device associated with a medical device in use by a patient, a patient communication from a remote server;
communicating the patient communication to the patient;
receiving an indication from the patient acknowledging receipt of the patient communication; and
communicating, from the external interface device, in response to the indication, an acknowledgement to the remote server.

17. The method of claim 16, comprising:
receiving, at the external interface device, physiological information from the medical device; and
communicating an indication of the physiological information from the external interface device to the remote server.

18. The method of claim 16, comprising processing the patient communication, wherein the processing the patient communication includes reprogramming the medical device in use by the patient, wherein some or all of the patient communication is used to reprogram the medical device.

19. The method of claim 16, comprising processing the patient communication, wherein processing the patient communication includes alerting the patient of the patient communication.

20. The method of claim 16, comprising displaying an indication of the patient communication to the patient.

21. The method of claim 20, wherein the indication is one or more of: an icon, a graphic, a sound, a light, or a vibration.

22. The method of claim 16, comprising receiving an indication from the patient acknowledging consent to perform a programming action.

23. The method of claim 22, wherein the programming action includes reprogramming the medical device using the external interface device.

24. The method of claim 16, comprising receiving an authenticated indication from the patient acknowledging at least one of receipt of the patient communication or consent to perform a programming action.

25. The method of claim 16, comprising authenticating the indication from the patient.

26. The method of claim 25, comprising using biometric information specific to the patient to perform the authenticating.

27. The method of claim 16, wherein receiving the indication from the patient acknowledging receipt of the patient communication includes receiving patient-specific authentication information that can be recorded for authentication purposes.

28. The method of claim 16, wherein the medical device in use by the patient is an implanted medical device.

29. A machine-assisted method comprising:
receiving, at a remote server, information from an external interface device that communicates with a medical device in use by a patient;
communicating, from the remote server to the external interface device, a patient communication to be received by the patient;
receiving, at the external interface device, a patient communication from the remote server;
communicating from the external interface device, the patient communication to the patient;
receiving, at the external interface device, an indication from the patient acknowledging receipt of the patient communication;
communicating from the external interface device, in response to the indication, a patient acknowledgement to the remote server; and
receiving, at the remote server, the patient acknowledgement to the patient communication from the external interface device.

30. The method of claim 29, comprising recording information about the patient acknowledgement.

31. The method of claim 29, comprising:
providing a user of the remote server, information from the external interface device received by the remote server; and
receiving from the user information to be included in the patient communication.

32. The method of claim 29, wherein the patient communication is an automatically generated response based, at least in part, on the information from the external interface device.

33. The method of claim 29, wherein the response communication includes one or more of: an email, a pager message, a voice message, a text message, or other electronically transmittable message.

34. The method of claim 29, comprising generating an alert if the patient acknowledgement is not received within a specified time period.

35. The method of claim 29, comprising authenticating the indication from the patient that acknowledged the receipt of the patient communication.

36. The method of claim 35, comprising using the patient's biometric information for authentication.

37. The method of claim 29, wherein receiving the indication from the patient acknowledging receipt of the patient communication includes receiving patient-specific authentication information that can be recorded for authentication purposes.

38. A system comprising:
an external interface device communicatively coupled to a patient medical device; and
a remote server communicatively coupled to the external interface device, wherein the remote server is adapted to receive information from the external interface device, and wherein the remote server is further adapted to communicate a patient communication to the external interface device, and wherein the remote server is further adapted to receive an acknowledgement communication from a patient indicating that the patient has received the patient communication.

39. The system of claim 38, comprising one or more sensor devices communicatively coupled to the external interface device.

40. The system of claim 39, wherein the information communicated to the remote server is related to the one or more sensor devices.

41. The system of claim 38, comprising an implantable cardiac function management device.

42. The system of claim 38, wherein the remote server is further adapted to record the acknowledgement communication.

43. The system of claim 38, wherein the external interface device is adapted to authenticate the patient's acknowledgement before communicating the acknowledgement to the remote server.

44. The system of claim 43, wherein the external interface device authenticates the patient's acknowledgement at least in part using the patient's biometric information.

45. The system of claim 38, wherein the external interface device is adapted to communicate an indication that the patient acknowledgement is authenticated.

46. The system of claim 38, wherein the patient medical device is an implanted medical device.
